(19) United States
(12) Patent Application Publication
(10) Pub. No.: US 2022/0313095 A1
(43) Pub. Date: Oct. 6, 2022

(54) THERMAL AND OPTICAL ANALYSES AND ASSESSMENTS

(71) Applicant: Intellisite, Ceres, CA (US)

(72) Inventors: Leandro Lichtensztein, Córdoba (AR); Mariano Garagiola, Córdoba (AR); Franco Zacco, Córdoba (AR); Jorge Campos, Modesto, CA (US); Mario Campos, Fort Mill, SC (US); Michael Knight, Greenville, SC (US); Ken Mills, Fort Mill, SC (US)

(73) Assignee: Intellisite

(21) Appl. No.: 17/220,673

(22) Filed: Apr. 1, 2021

(51) Int. Cl. A61B 5/01; A61B 5/1171; A61B 5/00

(52) U.S. Cl. CPC A61B 5/01; A61B 5/1176; A61B 5/7246; A61B 5/742; A61B 5/0017; A61B 5/0008; A61B 2562/0271

(57) ABSTRACT

A temperature assessment system aligns and analyzes optical sensor data and thermal sensor data to perform temperature assessments in a physical environment. The temperature assessment system may include facial detection and distance verification components, using a combination of machine learning and heuristics-based techniques to generate bounding boxes and perform individual assessments for multiple different people within the environment. Assessments may include temperature assessments, thermal uniformity assessments, and mask detection, and the results of the assessments may be used to transmit outputs to monitoring systems, provide alert notifications, and control various output devices based on the assessment status of the individual.

### BACKGROUND

Temperature scanning and/or mask detection devices may be used to verify the health safety of individuals at locations such as schools, airports, and large event venues. Temperature scanning devices can be used to detect individuals having abnormally high body temperatures, indicating potential fevers and illnesses, and automated mask detection devices may be deployed for safety screening and/or to comply with public health regulations. For instance, a temperature scanning device may identify individuals having a body temperature greater than an elevated temperature threshold (e.g., 100.4° F.), and such individuals may be prohibited from entering a building, boarding a flight, etc. Similarly, a mask detection device may perform a visual scan to identify individuals that are not wearing masks, and these individuals may be required to don masks before obtaining access to a building or other location.

Temperature scanning systems are often integrated into kiosks (or other standalone devices) that use thermal and/or infrared scanning technology to evaluate the body temperature of the person addressing the kiosk. Such temperature scanning systems are typically configured to scan only one person at a time, which may be slow and inadequate for scanning large numbers of individuals in a short time, and also may discourage social distancing by causing individuals to crowd or line up in front of the temperature scanning kiosks. In contrast, some temperature scanning systems deploy wide-angle temperature sensors and/or multiple sensors to perform temperature scanning of individuals over a larger area. These systems may scan a room, hallway, or other larger field of view, and may trigger an alert when detecting any heat source greater than a threshold temperature. However, temperature scanners covering larger areas are often susceptible to false positives, such as when an individual is carrying a hot beverage or a smartphone, or when the individual's clothing or hair are warmer than normal after recently being out in the sun.

Mask detection systems may use optical sensors (e.g., cameras) and/or infrared sensors to detect faces within a field of view, and then may analyze the sensor data to determine whether the individual is or is not wearing a mask. Certain mask detection systems may use machine-learning techniques and pattern matching to identify and classify object by shape within the field of view, detect the presence of faces, and identify masks. However, such mask detection systems are often error-prone, and may be susceptible to false positives and false negatives, due to the wide variety of face shapes, facial features, mask styles, etc. For instance, certain mask detection systems may mistake beards for masks, or may fail to detect masks with facial designs as well as transparent masks or face shields.

### BRIEF DESCRIPTION OF THE DRAWINGS

The detailed description is described with reference to the accompanying figures. In the figures, the left-most digit(s) of a reference number identifies the figure in which the reference number first appears. The use of the same reference numbers in different figures indicates similar or identical components or features.

FIG. 1 illustrates an example of a user interface provided by a temperature assessment system configured to perform temperature and mask assessments within a physical environment, in accordance with implementations of the disclosure.

FIG. 2 is a block diagram illustrating an example computing environment including a temperature assessment system, in accordance with implementations of the disclosure.

FIG. 3 is a block diagram illustrating an example architecture of a temperature assessment system configured to perform temperature and mask assessments based on optical and/or thermal sensor data, in accordance with implementations of the disclosure.

FIGS. 4A-4C are example diagrams illustrating techniques for performing temperature assessments, thermal uniformity assessments, and mask compliance assessments, and for determining control system outputs based on the assessments, in accordance with implementations of the disclosure.

FIG. 5 is a flow diagram illustrating an example process of performing temperature and/or mask assessments based on optical and thermal sensor data, in accordance with implementations of the disclosure.

FIG. 6 is a flow diagram illustrating an example process of controlling output systems based on evaluations of temperature and/or mask assessments, in accordance with implementations of the disclosure.

### DETAILED DESCRIPTION

As discussed above, conventional temperature scanning systems may be error prone and may include functional limitations that inhibit their usefulness in some environments. To address these and other limitations of conventional systems, this application describes techniques for aligning and analyzing optical sensor data and thermal sensor data to perform temperature assessments and mask detection assessments in physical environments. As described below, a temperature assessment system may determine a region of interest within the perception field (or field of view) of both an optical and thermal sensor systems. Within the region of interest, the temperature assessment system may perform facial detection and distance verification operations to identify individuals within an ideal temperature scanning range. The temperature assessment system then may perform temperature assessments on any individuals within the scanning range, along with thermal uniformity assessments to evaluate the quality of the temperature assessments, and mask detections to determine the compliance of the individuals with masking requirements.

Accordingly, the techniques described herein may provide improvements and technical advantages over conventional temperature scanning systems and/or mask detection systems. For instance, various implementations of temperature assessment systems described herein may align optical and thermal sensor image frames, to detect and assess individuals based on the combined optical and thermal data. As a result, the temperature assessment system may scan a larger perception field to separately detect and assess multiple different individuals within the same sensor perception field. The temperature assessment system thus provides the capability for faster scanning throughput of large numbers of people, which reduces delays and discourages the crowding and lack of social distancing of conventional temperature scanning kiosks.

The temperature assessment and mask detection techniques described herein also may use a combination of machine learning and heuristics-based operations, which analyze both the optical and thermal data to improve the quality of assessments and reduce errors. For example, the temperature assessment system may execute machine learning models for facial detection and bounding box generation, and a heuristics-based distance verification component, to detect and track individuals as they move through the perception field of the thermal sensors. For individuals within an optimal temperature scanning range, the temperature assessment system may execute machine learning and/or heuristics-based operations to perform temperature assessments, thermal uniformity assessments, and/or mask detection.

In some examples, the temperature assessment for an individual may include detecting a maximum temperature reading within a bounding box associated with the individual, and then a determining a thermal uniformity value to evaluate the temperature assessment. The thermal uniformity value may correspond to the number or percentage of temperature readings of the individual within a predetermined range (e.g., 3° F.) of the maximum temperature reading, and if the thermal uniformity value is below a uniformity threshold, the temperature assessment system may reject and/or reassess the temperature assessment for the individual. As described in more detail below, both the individual temperature assessments and the thermal uniformity assessments may execute various different machine learning and heuristics-based techniques, to improve the assessment accuracy and reduce errors. Additionally, the temperature assessment system may perform mask detection operations based on the combined analyses of both the optical and thermal sensor data, which may improve the results of the mask detection and reduces and/or eliminate false positive and false negative errors. In some examples, a mask detection analysis may be based entirely on the steam of data from the optical sensor system(s), and may be independent of (but might potentially correlate with) the thermal uniformity assessment. In other examples, a mask detection analysis may be based on aligned image data from multiple thermal and/or optical sensors.

The temperature assessment may be implemented including various combinations of components, including both machine learning and heuristics-based components, to perform temperature assessments and related operations the provide improvements over conventional systems. For instance, the temperature assessment system as described herein may include a thermal data component and optical data component respectively configured to receive streams of sensor data from thermal and optical sensor systems, along with an overlay component configured to align image of frames of the thermal and optical sensor data. The temperature assessment systems described herein also may include one or more of a facial detection component, a distance assessment component, a temperature assessment component, a thermal uniformity assessment component, or a mask assessment component, each of which may be configured to use machine learning models and/or heuristics to perform a specific subset of the temperature assessment operations.

In various examples, the temperature assessment system may evaluate the temperature assessments, thermal uniformity, and/or mask detection assessments, to control transmissions and other outputs to various output systems. For instance, the temperature assessment system may include a user interface through a which a monitoring system may stream of a video feed of the environment, including a graphical data overlay showing the temperature assessment results of individuals within the environment. The temperature assessment system also may output alerts and other notifications to the monitoring system and/or other user devices, including alerts for the detection of abnormally high body temperatures or non-compliance with masking requirements. In some examples, the temperature assessment system may control electronic access systems such as locks or badging system to restrict an individual's access to a location based on the temperature assessments of the individual. Additional output systems that may be controlled by the temperature assessment system can include facial recognition systems, facial blurring systems, data logging systems, video storage repositories, two-way audio capabilities with the sensor environment, and other output systems described below.

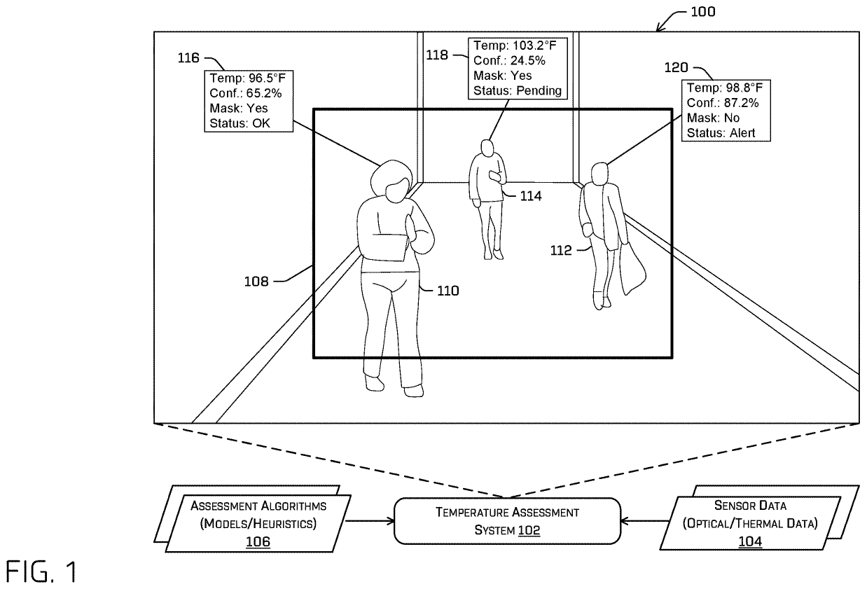

FIG. 1 shows an example of a user interface 100 provided by a temperature assessment system 102. As shown in this example, the temperature assessment system 102 is configured to detected individuals in an environment, and perform temperature and/or mask assessments of the individual. The temperature assessment system 102 may receive sensor data 104 from sensor system(s) operating in the environment, including optical and/or thermal sensor data. Using the optical and/or thermal sensor data, the temperature assessment system 102 may executed assessment algorithms 106, which may include machine-learning models and/or heuristics-based algorithms, to perform temperature assessments and mask detection assessments on individuals within the environment. In some examples, the temperature assessment system 102 may output a user interface 100, via a monitoring system or other client computing device, to allow administrators or other users to visually monitor an environment and observe the results of temperature and mask assessments in real-time as individuals proceed through the environment. The physical environment depicted in user interface 100 may represent an entrance to a school, office building, event venue, or the like. As described below, various sensor systems may operate within the environment to capture sensor data 104 and provide the sensor data to the temperature assessment system 102. However, the temperature assessment system 102 (and/or the monitoring systems or other client devices) need not operate within the same environment as the sensor systems, but may operate at different locations and/or networks.

In some examples, the user interface 100 may include a video feed of a physical environment, along with graphical overlays providing the results of assessments performed by the temperature assessment system 102 for any individual detected within the physical environment. The physical environment depicted in the user interface 100 may be an entryway, hallway, or corridor, through which users may proceed to gain access to a building or other restricted area. In some cases, the temperature assessment system 102 may determine and label a region of interest (ROI) 108 within the user interface 100. The temperature assessment system 102 may detect and perform assessments for individuals within the ROI 108, but may be configured not to detect or assess any individuals outside of the ROI 108. In some instances, the regions of the user interface 100 outside of the ROI 108 may be obscured (e.g., shaded or blurred) by the temperature assessment system 102. Although the ROI 108 shown in this example is a square ROI in the center of the field of view of the user interface, in various other examples the size, shape, and/or position of the ROI 108 may be dynamically determined by automated processes and/or by users of the temperature assessment system 102. For instance, the ROI 108 may be determined based on the usable or optimal perception range(s) of the sensor systems operating in the environment. As an example, the temperature assessment system 102 may determine the ROI 108 as an overlapping region between the fields of view of one or more optical sensors and/or thermal sensors operating in the environment. Additionally or alternatively, the temperature assessment system 102 may configure the ROI 108 based on the size and layout of the physical environment being scanned. For example, the size, shape, and location of an ROI 108 may be determined by the temperature assessment system 102 so that it will detect and assess all individuals approaching an entrance to a restricted area (e.g., a door or hallway), but will not assess individuals moving away from the restricted area, moving toward a different entrance, etc.

A user interface output by the temperature assessment system 102 may include an augmented reality (AR) representation of a physical environment. As shown in this example, the user interface 100 includes a video feed of an area, in which three individuals 110, 112, and 114 are shown within the ROI 108. For each individual in the ROI 108, the user interface 100 also includes an AR label 116, 118, and 120 to display the assessment data and status of the individuals. As described below in more detail, user interfaces are optional and need not provided by the temperature assessment system 102 in certain implementations. However, in this example, the user interface 100 illustrates different types of assessments and various functionality of the temperature assessment system 102, including detecting, tracking, and assessing multiple different individuals within a physical environment at the same time. In some examples, temperature assessments and/or mask assessments of individuals may be based on a single frame of optical and/or thermal sensor data. Additionally, in some cases the temperature assessment system 102 may detect and track multiple individuals as they move through the physical environment, and may perform multiple assessments for each individual at different times and/or locations within the environment.

The temperature assessment system 102 may perform different types of assessments and/or combinations of assessments for the individuals within the ROI 108. As shown in this example, the user interface 100 includes AR labels 116-120 showing the results of the temperature assessments, the confidence values associated with the temperature assessments (e.g., based on thermal uniformity assessments), and mask compliance assessments for each of the detected individuals 110-114. The temperature assessment system 102 may analyze data sensor data associated with each individual 110-114 separately, using machine-learning models and/or heuristics to determine the assessment values for each of the individuals 110-114. The temperature assessment system 102 also may use the assessment results for each individual to determine a status value (or multiple statuses) for each individual 110-114 within the ROI 108. As described in more detail below, the statuses determined for an individual may be associated with various classifications or categories that may cause the temperature assessment system 102 to perform output actions, such as triggering alerts at a monitoring system, transmitting notifications to other user devices, granting or denying access to restricted areas via electronic access systems, and/or may initiate other functionalities in other output systems.

In user interface 100, individual 110 is shown as the closest person to the optical and/or thermal sensors within the environment. As indicated by the AR label 116, the temperature assessment system 102 has assessed individual 110 to have a temperature of 96.5° F., with a confidence value of 65.2%, and a successful mask verification. As a result, the temperature assessment system 102 has determined a status of “OK” for individual 110. In this example, the “OK” status for individual 110 may indicate that the individual 110 will be permitted to proceed into a restricted location without a delay or further assessments, or that no alerts or notifications will be sent out by the temperature assessment system 102, etc. Although in this example a confidence value is determined for the individual 110 whose temperature (e.g., 96.5° F.) is within a normal temperature range, in other examples a confidence value based on a thermal uniformity assessment might be determined only for the detected individuals having temperatures above and/or below a normal temperature range.

Individual 112 is the second closest person to the optical and thermal sensors in the user interface 100. As indicated by the AR label 118, the temperature assessment system 102 has assessed individual 112 to have a temperature of 98.8° F., with a confidence value of 87.2%, and a failing mask verification. Due to the failure of individual 112 to comply with a mask requirement, the temperature assessment system 102 has determined a status of “Alert” associated with the individual 112. In this example, the “Alert” status may trigger one or more alerts notifications to client devices, and/or may cause the temperature assessment system 102 to prevent the individual 112 from accessing a restricted location.

Individual 114 is the farthest person from the optical and thermal sensors in the user interface 100. As indicated by the AR label 120, the temperature assessment system 102 has assessed individual 114 to have a temperature of 103.2° F., with a confidence value of 24.5%, and a successful mask verification. In this example, due to the combination of the high body temperature and the low confidence value, the temperature assessment system 102 has assigned a status of “Pending” for the individual 114. As described below, the “Pending” status or other inconclusive data assessments may cause the temperature assessment system 102 to reassess the body temperature of the individual 114, which may be done using various different techniques as the individual 114 moves through the ROI 108 in the environment.

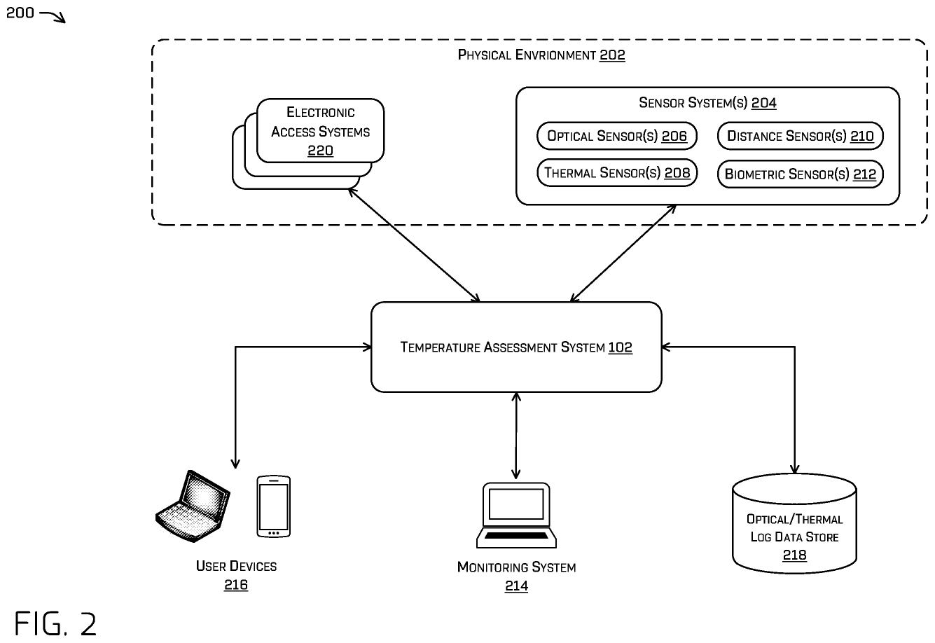

FIG. 2 shows an example computing environment 200 including a temperature assessment system 102 in communication with various related computing devices and systems. In this example, the temperature assessment system 102 receives sensor data from sensor systems 204 operating within a physical environment 202. As noted above, the physical environment 202 may include any environment within which individuals may be detected and assessed, including but not limited to schools, office buildings, airports, event venues, and the like. The sensor system(s) 204 may include one or more optical sensors 206 (e.g., cameras) configured to capture and analyze visual data, and/or thermal sensors 208 configured to capture and analyze temperature data within the physical environment 202 using infrared or thermal scanning technology. As noted above, the temperature assessment system 102 may use a combination of optical data and thermal data to perform the temperature assessments and/or mask assessments described herein. Additionally, in various implementations the physical environment 202 may include additional sensor systems 204 configured to collect and provide additional types of sensor data to the temperature assessment system 102. For instance, distance sensor(s) 210 (e.g., LIDAR or RADAR systems) may determine the distances between the sensor system(s) 204 and various individuals or other objects in the physical environment 202, and may provide the distance data to the temperature assessment system 102 to be used in performing temperature and mask assessments. Biometric sensors 212 (e.g., fingerprint scanners, voiceprint scanners, retinal scanners, etc.) also may be installed within the physical environment 202, and may be configured to provide biometric data for the individuals detected within the environment.

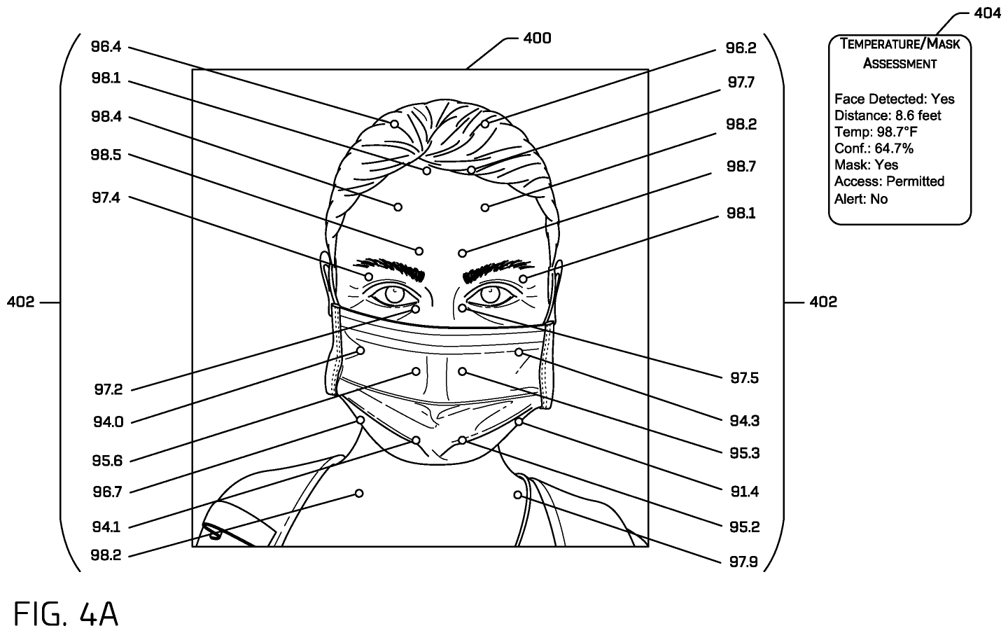

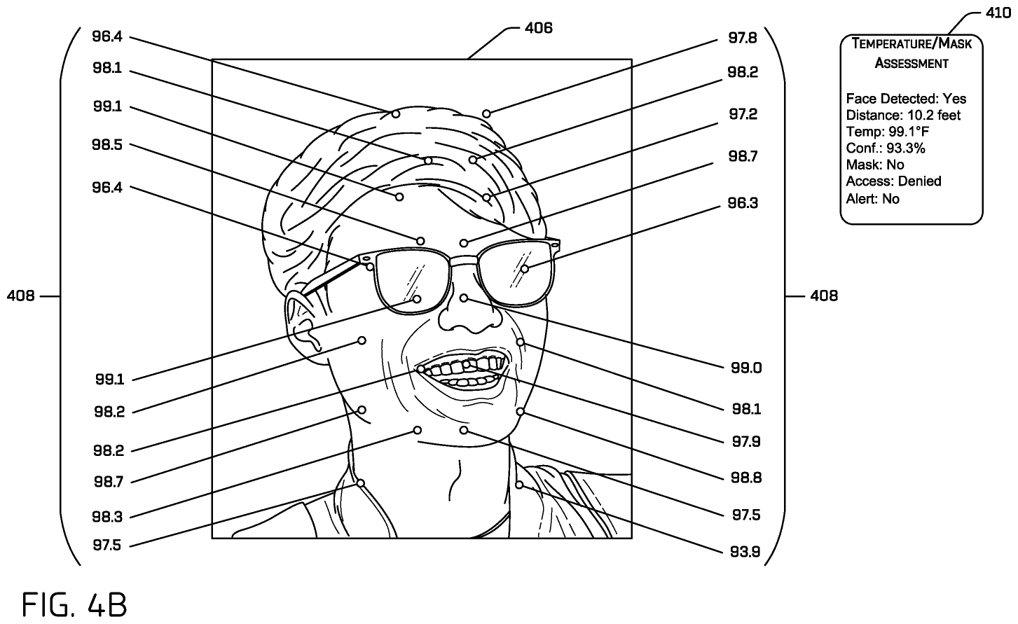

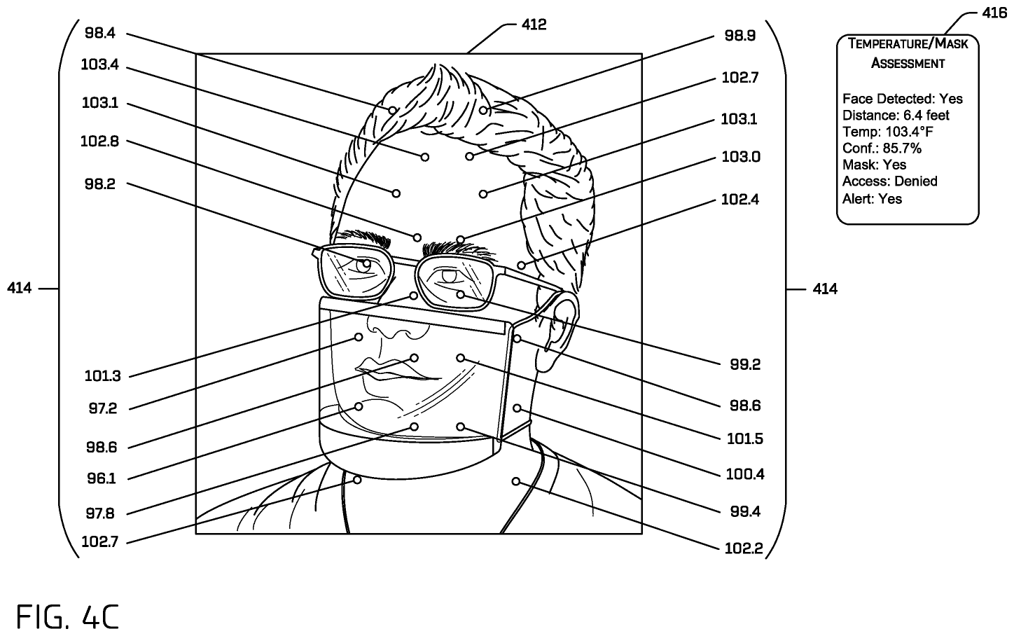

In this example, the temperature assessment system 102 receives data from sensor system(s) 204, and uses the sensor data to perform various assessments of individuals within the physical environment 202. The various assessments may include any of the types of temperature assessments, thermal uniformity assessments, and/or mask assessments described below. Prior to performing an assessment of an individual, in some instances the temperature assessment system 102 may identify one or more ROIs within the physical environment 202, and may use facial detection to detect individuals within the ROIs. In some cases, the temperature assessment system 102 also may use data from distance sensors 210 to determine when an individual in the ROI is within a predetermined distance from the sensor systems 204, such as an optical scanning range of the thermal sensor(s) 208. When the temperature assessment system 102 detects the individual via facial detection, within the ROI, and/or within the predetermined distance from the sensor systems 204, the temperature assessment system 102 may automatically trigger one or more assessments on the individual. As described below in more detail, the various types of temperature assessments, thermal uniformity assessments, and/or mask assessments may use machine-learning models and/or heuristics-based algorithms, which may use as input data any combination of the sensor data received from sensor systems 204. In some examples, the temperature assessment system 102 also may receive additional data, such as the previous temperature readings from a temperature log, and/or other data associated with an individual, to use as input data to the temperature assessments and/or mask assessments. Examples of various machine learning models and/or heuristics algorithms used by the temperature assessment system 102 are described in more detail below in reference to FIGS. 4A-6.

After performing temperature and/or mask assessments on an individual within the physical environment 202, the temperature assessment system 102 may analyze the results of the assessments to determine one or more output actions to perform within the computing environment 200. For instance, based on the assessment results for an individual, the temperature assessment system 102 may be configured to output data to a monitoring system 214, generate and transmit notifications to user devices 216, and/or store the results of individual assessments and related data within a log data store 218. Additionally or alternatively, the temperature assessment system 102 may be configured to perform actions using devices within the physical environment 202 based on the assessment results of individuals in the environment, for instance, by controlling the sensor system(s) 204, electronic access systems 220, and/or other devices within the physical environment 202.

A monitoring system 214 may be configured to observe, output, and/or analyze the results of the assessments performed by the temperature assessment system 102 of individuals within the physical environment 202. In some examples, the monitoring system 214 may provide a user interface based on the assessment data from temperature assessment system 102, that allows an administrator, security officer, or other user to monitor the physical environment 202 in real-time or near real-time. For instance, the temperature assessment system 102 may provide a user interface to the monitoring system 214, including a video feed of the physical environment 202, along with AR labels to display the assessment results for individuals passing through the ROI of the physical environment 202. Such user interfaces also may display individual statuses and/or alerts, and/or may allow users at the monitoring system 214 to register for and receive notifications, configure the sensor systems 204, or control other devices within the physical environment 202.

The monitoring system 214 may be fully or partially automated in some implementations, and need not output a graphical user interface or interact with a human user to monitor the physical environment 202. For instance, the monitoring system 214 may be implemented as a separate system or may be integrated into the temperature assessment system 102, and may execute automated operations to perform actions in response to the assessments of individuals performed by the temperature assessment system 102. Additionally, although only one temperature assessment system 102 and one monitoring system 214 are depicted in this example, in some cases a temperature assessment system 102 may monitor multiple different physical environments, and/or a monitoring system 214 may be associated with multiple different temperature assessment systems. In such examples, a single monitoring system 214 therefore may provide user interfaces and/or automated monitoring capabilities simultaneously at multiple different physical environments (e.g., multiple entryways to a restricted location, etc.).

In some examples, a monitoring system 214 may be configured to perform an action in response to an individual failing a temperature or mask assessment, and/or an alert being generated by the temperature assessment system 102. When the monitoring system 214 provides a user interface, it may display data indicating the failure or alert on the user interface, and may allow users of the monitoring system 214 to perform various actions responsive to the alert/failure. For a monitoring system 214 using automated monitoring capabilities, it may initiate rules-based actions automatically in response to certain results of individual assessments performed by the temperature assessment system 102. As an example, in response to an assessment failure or alert generated by the temperature assessment system 102, the monitoring system 214 may open an audio and/or video channel to the physical environment 202, and may provide specific instructions to the individual that triggered the alert/failure, such as instructions to the individual to put on a face mask before entry, or to proceed to a separate kiosk for a temperature scan, ID scan, biometric scan, etc.

As another example, in response to assessment failures or alerts generated by the temperature assessment system 102, the monitoring system 214 may record the alert/failure in a log data store 218, along with data identifying the individual that triggered the alert/failure and the assessment results. In some cases, the monitoring system 214 may be configured to blur/unblur the faces of individuals, either within a user interface or for recording image/video log data, based on the results of individual assessments. For instance, as a configurable privacy feature, the monitoring system 214 may automatically blur the faces detected within the ROI. However, when an individual fails a temperature or mask assessment, the monitoring system 214 may unblur the individual's face and may initiate a facial recognition operation to identify the individual. The monitoring system 214 also may record image and/or video data of the individual, biometric data of the individual, or a badge/ID number of the individual as they pass through a badging system, to determine the identity of the individual that caused the failure/alert. In various examples, the temperature assessment system 102 and/or monitoring system 214 may conditionally transmit individual assessment results (e.g., temperature or mask assessment failures or alerts), or may transmit all individual assessment results, to the log data store 218. Although the log data store 218 is shown external to the temperature assessment system 102 in this example, in some implementations the log data store 218 may be stored in an internal memory of the temperature assessment system 102.

In some examples, the temperature assessment system 102 and/or monitoring system 214 also may support registration for assessment notifications. As described below, assessment notifications may be based on a set of notification conditions, and may be associated with one or more output channels. The output channel for a notification may include a user device 216 and/or network address (e.g., email address, mobile number, IP address, etc.), an alert/notification via user interface on the monitoring system 214, and/or storing notification data on within a log data store 218. When the set conditions for a notification is satisfied, the temperature assessment system 102 and/or monitoring system 214 may generate the notification including the relevant information (e.g., time, individual identifier, assessment results, etc.) and transmit the notification to the output channel(s).

Notification conditions may be based the results of the assessments performed by the temperature assessment system 102. For instance, a notification may be triggered when the temperature assessment system 102 detects an individual in the physical environment 202 that has failed to comply with a mask requirement, and/or when the temperature measurement for an individual (and/or the associated confidence value) meets or exceeds one or more thresholds. Additionally or alternatively, notification conditions may be associated with the detection of a particular individual within the physical environment 202. For such notifications, the temperature assessment system 102 and/or monitoring system 214 may use facial recognition, voiceprint identification, biometrics, badge/ID verification, and/or other user verification techniques to determine the identity of the individuals passing through the physical environment 202, and then may generate notifications in response to detecting specific individuals. In some cases, any detection of a particular individual may trigger a notification, and in other cases only a detection of the particular individual having certain temperature or mask assessment results may trigger a notification.

As noted above, the temperature assessment system 102 and/or monitoring system 214 also may be configured to control electronic devices within the physical environment 202 based on the results of the assessments. In some examples, the temperature assessment system 102 may control electronic access systems 220 to permit or deny an individual with access to a restricted area based on the mask and/or temperature assessment of the individual. For instance, the physical environment 202 may be an entryway to a location (e.g., building, room, terminal, hall, etc.) that is access-restricted by a locking door, badging system, and/or guard station, etc. As an individual approaches the restricted area, he/she may pass through the physical environment 202, during which the temperature assessment system 102 may perform one or more of the mask and/or temperature assessments described herein. If the individual fails the assessments (e.g., mask non-compliance, temperature exceeds a threshold, etc.), then the temperature assessment system 102 and/or monitoring system 214 may transmit instructions to an electronic access system 220 to prevent the individual from accessing the restricted area. In some instances, the temperature assessment system 102 may identify the particular individual, and the access restriction instructions transmitted to the electronic access system 220 may include an identifier (e.g., name, employee number, badge number, etc.) of the restricted individual. As discussed above, the temperature assessment system 102 and/or monitoring system 214 also may perform other actions when restricting access to a location, such as providing information and instructions to the restricted individual (or nearby security personnel) via an audio or video channel, to direct the individual to a separate location for further assessment. The temperature assessment system 102 and/or monitoring system 214 also may activate lights and audible alarms within the physical environment 202, for example, to signal an alert or an unauthorized entry to a restricted area.

The computer systems and devices shown in computing environment 200 may be implemented using various computer architectures, including computer servers, workstations, desktop or laptop computers, tablet computers, network appliances, mobile devices (e.g., smartphones, etc.), and/or other computing devices. Any or all of the computing systems described herein may be implemented as virtual machines and/or as software services. In some examples, the sensor systems 204, user devices 216, electronic access systems 220 and/or other computing devices described herein may be implemented as mobile devices and/or Internet-of-Things (IoT) devices configured to communicate with the temperature assessment system 102 via wireless networks and wireless technologies.

Additionally, although the computing environment 200 in this example depicts several distinct computer systems and devices, the techniques described herein may be implemented in various different computing environments and architectures. For instance, any of the computer systems or devices described herein may be integrated into the temperature assessment system 102. Any of the various computer systems or devices may be implemented within the same computer server using the same hardware infrastructure and/or within the same datacenter. For instance, the temperature assessment system 102, monitoring system 214, and log data store 218 may be implemented as components/services in a single computer server operating within the physical environment 202. In other examples, temperature assessment system 102, monitoring system 214, and/or log data store 218 may operate on separate physical and/or virtual servers within a cloud computing environment or datacenter that is outside of the physical environment 202.

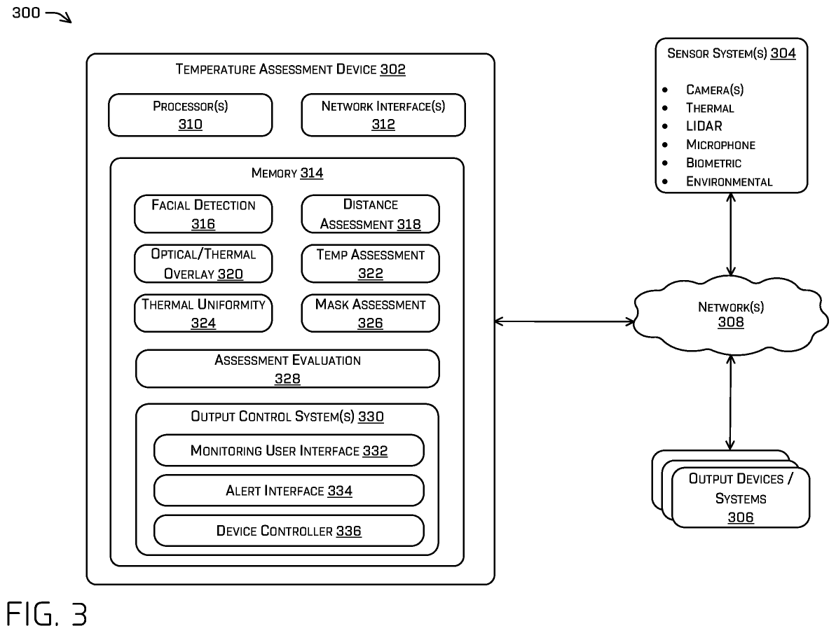

FIG. 3 depicts a block diagram illustrating an example system 300 for implementing various techniques discussed herein. In at least one example, the system 300 may include a temperature assessment device 302 configured to perform temperature and/or mask assessments of individuals based on the data received from sensor systems 304. The temperature assessment device 302 also may control output devices and/or systems 306 over one or more networks 308, based on the assessment results. In some implementations, the temperature assessment device 302 may be similar or identical to the temperature assessment system 102 described herein, and may include any or all of the features and functionalities discussed in connection with the temperature assessment system 102 and/or its related systems. The temperature assessment device 302 shown in this example may be implemented using one or more computer servers and/or other computing devices, including one or more processor(s) 310 and memory 314 storing components configured to perform the various functionalities described herein. In various implementations, the temperature assessment device 302 may be implemented as a standalone computer system, a distributed system (e.g., client-server or service-based architecture, one or more virtual machine(s), and/or software services, which may operate within a datacenter and/or within a cloud computing environment. The temperature assessment device 302 in this example also includes one or more network interface(s) 312 (e.g., communication devices and/or modems) to allow the temperature assessment device 302 to exchange data with the sensor system(s) 304 and/or output devices/systems 306, over one or more communication networks 308.

The one or more sensor system(s) 304 may be configured to capture various sensor data associated with a surrounding physical environment (e.g., physical environment 202). In at least some examples, the sensor system(s) 304 may include cameras or other optical data sensors (e.g., RGB, IR, intensity, depth, etc.), thermal sensors that use thermal and/or infrared scanning technology to perform temperature scans, distance sensors (e.g., lidar sensors, radar sensors, sonar sensors, etc.), biometric sensors (e.g., fingerprint scanners, retinal scanners, etc.), and/or environmental sensors (e.g., microphone sensors, ambient temperature sensors, humidity sensors, light sensors, pressure sensors, etc.). In some examples, the sensor system(s) 304 may include multiple instances of each type of sensors. For instance, a physical environment 202 may include multiple cameras and/or multiple thermal sensors disposed at different locations in the environment. In various examples, the sensor system(s) 304 may capture and provide sensor data to the temperature assessment device 302, and/or may perform one or more sensor data preprocessing operations, filtering operations, analysis operations, etc., before transmitted the sensor data to the temperature assessment device 302.

As noted above, in some cases the sensor systems 304 also may include audio and/or visual emitters to communicate with individuals in the physical environment 202. Such emitters may include speakers, lights, signs, display screens, touch screens, and the like. By way of example and not limitation, such emitters may include lights to direct the path of the individual based on their assessment results, and/or speakers to audibly communicate with the individual.

The network interface(s) 312 may include physical and/or logical interfaces for connecting the temperature assessment device 302 to other computing devices or network(s) 308 (e.g., the Internet). For example, the network interface(s) 312 can enable Wi-Fi-based communication such as via frequencies defined by the IEEE 802.11 standards, short range wireless frequencies such as Bluetooth, cellular communication (e.g., 2G, 3G, 4G, 4G LTE, 5G, etc.), satellite communication, dedicated short-range communications (DSRC), or any suitable wired or wireless communications protocol that enables the respective computing device to interface with the other computing device(s). In at least some examples, the network interface(s) 312 may comprise the one or more modems as described above.

As noted above, in response to the results of temperature and/or mask assessments of individuals within a physical environment, the temperature assessment device 302 may output instructions to control one or more output devices/systems 306. The output devices/systems 306 may include electronic access systems (e.g., automated locks, badging systems, etc.), communication devices (e.g., output display screens, video/audio emitter systems), additional sensor systems (e.g., cameras, biometrics, etc.), or remote user devices to allow users to monitor assessment results and/or receive alerts or notifications. Each of the output devices/systems 306 can include one or more processor(s) and memory communicatively coupled with the one or more processor(s). The memory can store one or more modules to perform various functionalities of the devices/systems. Additionally, the output devices/systems 306 also may include network interfaces to enable communication by the respective device or system with one or more other local or remote computing device(s).

In the illustrated example, the memory 314 of the temperature assessment device 302 stores a number of components configured to work in conjunction to perform temperature and/or mask assessments of individuals detected based on the data received from the sensor systems 304. Such components may include but are not limited to a facial detection component 316, a distance assessment component 318, a thermal/optical overlay component 320, a temperature assessment component 322, a thermal uniformity assessment component 324, and a mask assessment component 326.

The facial detection component 316 may be configured to continually monitor a visual data stream (e.g., a video feed) of an area to detect and/or recognize human faces within the area. For example, to detect that an individual has entered an ROI within a physical environment 202, the facial detection component 316 may receive and analyze data from one or more cameras within the environment and/or other types of sensors within sensor systems 304 (e.g., lidar/radar, infrared, microphones, biometrics, etc.) that may be used to detect or confirm the presence and location of the individual. In some instances, the facial detection component 316 may include separate facial detection and facial recognition subcomponents. Facial detection may be used in some cases without facial recognition, to detect that an individual is within an area without identifying the individual, thereby provided additional privacy and data security.

The distance assessment component 318 may use various sensor data (e.g., image data, lidar or radar data, etc.), to determine the locations of any individuals detected within the physical environment, and/or the distances between the individuals and other individuals or objects in the environment. For instance, the distance assessment component 318 may be used to determine the distance between an individual and the thermal sensors that will be used to measure the body temperature of the individual. In some examples, the temperature assessment device 302 may perform assessments on an individual only when that individual is determined to be within an optimal distance range from the thermal sensors and/or cameras in the environment.

The thermal/optical overlay component 320 may be configured to receive and overlay separate thermal and optical data streams received from the sensor systems 304. As noted above, certain assessments described herein may use both thermal data and optical data, and synchronization of the thermal and optical data may provide improved assessment capabilities and accuracy. In some examples, the thermal/optical overlay component 320 may overlay the thermal stream (e.g., one or more thermal sensor image frames) received from the thermal sensors, on top of the optical stream (e.g., one or more optical sensor image frames), using an affine transformation to align the image frames.

The temperature assessment component 322 may use the data from the thermal sensors (and/or other sensor data) within sensor systems 304 to measure the body temperature of the individuals detected in the environment. In some examples, the temperature assessment component 322 may perform a temperature assessment for an individual by determining a bounding box associated with the individual and selecting a maximum temperature reading from within the bounding box. As described below, in various other examples the temperature assessment component 322 may use different temperature assessment techniques, including machine learning and/or heuristics-based techniques.

The thermal uniformity assessment component 324 may be associated with and/or integrated into the temperature assessment component 322 in some examples. As discussed below, in some cases the thermal uniformity assessment component 324 may determine thermal uniformity metrics associated with the body temperature measurements performed by the temperature assessment component 322. The thermal uniformity metrics may provide an indication of a confidence level for the body temperature measurements, and as described below may be used to determine when to reassess a body temperature, when to use a different temperature assessment technique, when to control certain output devices/systems, etc.

The mask assessment component 326 also may be associated with and/or integrated into the temperature assessment component 322 in some examples. As described below, the mask assessment component 326 may be configured to use optical sensor data, thermal sensor data, and/or various other sensor data from sensor systems 304 to determine whether an individual detected within the environment is wearing a facemask and/or is in compliance with a mask requirement. As described below, the mask assessment component 326 also may use various different mask assessment techniques, including machine learning and/or heuristics-based techniques.

In the illustrated example, the memory 314 also stores an assessment evaluation component 328 configured to evaluate the results of individual temperature assessments and/or mask assessments, and to determine actions to be performed based on the individual assessment results. In some cases, the assessment evaluation component 328 may use one or more output control systems 330 to transmit data or instructions to control external devices/systems. In this example, the output control systems 330 include a monitoring user interface component 332 configured to provide a monitoring user interface to an external monitoring system 214 based on the assessment results, an alert interface component 334 configured to provide an interface by which user devices 216 may register for and receive notifications based on the assessment results, and a device controller 336 which may transmit instructions to control various external devices/systems based on the assessment results. As described above, the device controller 336 may be used to transmit control instructions to external devices or systems such as electronic access systems (e.g., automated locks, badging systems, etc.), communication devices (e.g., output display screens, video/audio emitter systems), additional sensor systems (e.g., cameras, biometrics, etc.), or remote user devices, based on the temperature and/or mask assessment results. In some examples, device controller 336 may include an IoT controller configured to receive data from and transmit control instructions to external IoT devices via the network(s) 308.

The processor(s) 310 of the temperature assessment device 302 may be any suitable processor capable of executing instructions to process data and perform operations as described herein. By way of example and not limitation, the processor(s) 310 can comprise one or more Central Processing Units (CPUs), Graphics Processing Units (GPUs), or any other device or portion of a device that processes electronic data to transform that electronic data into other electronic data that can be stored in registers and/or memory. In some examples, integrated circuits (e.g., ASICs, etc.), gate arrays (e.g., FPGAs, etc.), and other hardware devices can also be considered processors in so far as they are configured to implement encoded instructions.

The memory 314 of the temperature assessment device 302 may include non-transitory computer-readable media. The memory 314 can store an operating system and one or more software applications, instructions, programs, and/or data to implement the methods described herein and the functions attributed to the various systems. In various implementations, the memory 314 can be implemented using any suitable memory technology, such as static random-access memory (SRAM), synchronous dynamic RAM (SDRAM), nonvolatile/Flash-type memory, or any other type of memory capable of storing information. The architectures, systems, and individual elements described herein can include many other logical, programmatic, and physical components, of which those shown in the accompanying figures are merely examples that are related to the discussion herein.

In some instances, aspects of some or all of the components discussed herein can include any models, algorithms, and/or machine learning algorithms. For example, in some instances, the components in the memory 314 can be implemented as a neural network.

FIGS. 4A-4C show three images of three different individuals, along with example temperature readings for each individual at different locations, and the results of temperature and mask assessments associated with the individuals. As described below, FIGS. 4A-4C illustrate various assessment techniques that may be performed by a temperature assessment system 102, including examples of temperature assessments, thermal uniformity (and/or confidence) assessments, and/or mask assessments. It can be understood in the context of this disclosure that the assessment techniques described in connection with FIGS. 4A-4C are for illustrative purposes, and that additional or alternative techniques for performing temperature assessments, thermal uniformity assessments, and/or mask assessments may be used in other examples.

FIG. 4A shows a first image 400 depicting an individual, a first set of temperature readings 402, and a first information box 404 containing assessment results and related data associated with the individual in the first image 400. Similarly, FIG. 4B shows a second image 406, a second set of temperature readings 408, and a second information box 410, and FIG. 4C shows a third image 412, a third set of temperature readings 414, and a third information box 416. In these examples, the sets of temperature readings may represent temperature measurements performed by one or more thermal sensors 208 within the physical environment 202. In some examples, the thermal sensors 208 may use thermal and/or infrared technology that permits the temperature assessment system 102 to receive multiple temperature measurements, based on the same optical/thermal images, for different target locations on or around the individuals depicted in the images. For instance, each of the images in FIGS. 4A-4C shows twenty-two (22) separate temperature measurement points. However, it can be understood in the context of this disclosure that different numbers of temperature measurements may be taken for a thermal/optical image in other examples, depending on the thermal sensor specifications and configuration, and/or the distance between the thermal sensors 208 and the individual.

In FIGS. 4A-4C, the individuals depicted in the images 400, 406, and 412 may represent individuals that were detected by a facial detection component 316 within the ROI of a physical environment being monitored by the temperature assessment system 102. Additionally, the distance assessment component 318 may be used to determine distances between each of the individuals depicted and the respective thermal sensors 208. As noted above, in some examples the temperature assessment system 102 may perform assessments on only those individuals within a predetermined range from the sensor system(s) 204, such as an optimal distance from the thermal sensors 208 to provide more accurate temperature measurements.

In some examples, the temperature assessment system 102 may determine a bounding box associated with an individual that has been detected and/or distance-verified within the ROI of an environment. For instance, the sizes and shapes of images 400, 406, and 412 may represent rectangular bounding boxes determined by the temperature assessment system 102, in which the size and shape of the bounding boxes was determined so that they would include the head and neck region of the individuals for optimal temperature assessments. In other examples, the temperature assessment system 102 may determine non-rectangular bounding boxes of various other sizes and shapes. For instance, the temperature assessment system 102 may use the visual sensor data, thermal sensor data, and/or lidar/radar sensor data to determine bounding boxes that correspond to the outline (or silhouette) of the detected individual, so that temperature measurements of the space around the individual are excluded from the temperature and/or mask assessment processes. As described below, the temperature assessment system 102 may use the bounding boxes to determine which of the temperature measurements received from the thermal sensors 208 are analyzed when performing the assessments.

In the examples depicted in FIGS. 4A-4C, the temperature assessment system 102 may perform temperature and/or mask assessments of individuals based on a single frame of sensor data (e.g., an aligned and synchronized optical/thermal sensor data frame). Although assessments of individuals may be performed based on a single data frame representing a single point in time, in other examples, the temperature assessment system 102 may perform a temperature assessment and/or mask assessment based on multiple data frames corresponding to different times and/or different distances of the individual from the sensor systems 204. For instance, if the temperature assessments performed for an individual at two different times result in different temperatures and/or different thermal uniformity values, the temperature assessment system 102 may average or otherwise combine the assessment results, and/or may determine that additional temperature assessments are to be performed as the individual moves closer to the sensor systems 204. Additionally, in this example the thermal sensors 208 provide temperature data at a precision of 0.01° F. However, the thermal sensors 208 may provide more or less precision in other examples, and the temperature precision may vary based on the distance between the individual and the thermal sensors 208 and/or based on environmental conditions such as the ambient temperature, humidity, light, wind, etc., in the physical environment.

As shown in FIGS. 4A-4C, certain temperature measurements within the sets of temperature readings 402, 408, and 414 may measure the temperature at a point on the skin of the individual, while others may measure the temperature at points on the individual's hair, clothing, jewelry, etc., or may measure the ambient temperature near the individual. In some examples, the temperature assessment system 102 may be configured to identify the measurement points on the skin of the individual, and/or at particular locations on the individual that provide the most accurate body temperature readings, such as the forehead, neck, and upper torso. For instance, the temperature assessment system 102 may use facial and body detection algorithms, and/or machine-learning models, to determine a precise size, shape, and location of the face and neck of the individual. Additional algorithms and/or machine learning model may be used to identify the individual's hair, clothing, facial hair, jewelry, or other foreign objects, etc. Based on these locations, the temperature assessment system 102 may determine certain temperature measurement points (e.g., on the individual's forehead or neck) that are likely to provide more accurate body temperatures for the individual, and other measurement points (e.g., on the individual's hair, nose, mask, or clothing, etc.) that are unlikely to provide accurate body temperatures. Whenever the temperature assessment system 102 determines measurement points that are more or less likely to provide accurate body temperatures, it may bias or weight these temperature measurements, and/or exclude other temperature measurements, in any of temperature assessment techniques described herein, to account for the higher or lower quality of the particular temperature measurement points.

When performing a temperature assessment, the temperature assessment component 322 may use the set of temperature readings associated with an individual (e.g., within the bounding box for the individual), to determine a body temperature to be assigned to the individual. In the examples shown in FIGS. 4A-4C, the temperature assessment component 322 may determine a temperature for an individual based on the maximum temperature reading within the set of temperature readings for the individual. In such examples, the temperature assessment component 322 may determine that the maximum temperature reading within the bounding box may be more likely to correspond to a portion of the individual's skin that provides accurate body temperature readings, as opposed to other portions of the individual's skin, clothing, or hair that may provide lower and less accurate temperature readings. Accordingly, in FIG. 4A the temperature assessment component 322 has determined a temperature assessment value of 98.7° F., which is the highest temperature reading within the first set of temperature readings 402 for the individual in image 400. Similarly, in FIG. 4B the temperature assessment component 322 has determined a temperature assessment value of 99.1° F., which is the highest temperature reading within the second set of temperature readings 408 for the individual in image 406, and in FIG. 4C the temperature assessment component 322 has determined a temperature assessment value of 103.4° F., which is the highest temperature reading within the third set of temperature readings 414 for the individual in image 412.

In other examples, the temperature assessment component 322 might not use the maximum temperature reading within the bounding box to assess an individual's temperature, but may use additional or alternative techniques for temperature assessment. In some cases, the temperature assessment component 322 may average some or all of the temperature readings within the bounding box to determine a temperature assessment for an individual. Additionally or alternatively, the temperature assessment component 322 may select a maximum or average temperature reading after excluding any readings that fall outside of a predetermined temperature range. For instance, the temperature assessment component 322 may exclude any temperature readings above a maximum threshold (e.g., 110.0° F.) and below a minimum threshold (e.g., 90.0° F.), as these may be assumed to be erroneous readings or readings of something other than the individual's skin. Additionally, various averaging techniques may average all of the temperature readings within the bounding box, or may average a subset of the temperature readings (e.g., readings within a predetermined temperature range, readings within an N-degree range from the maximum, readings at certain locations visually identified on the individual, readings that exclude other locations visually identified on the individual, etc.).

In addition to performing temperature assessments of individuals, in some cases the temperature assessment system 102 may perform thermal uniformity assessments based on the set of temperature readings associated with the individual. As noted above, thermal uniformity assessments may be associated with temperature assessments, and may be used to determine a confidence values associated with the temperature assessments. Various different techniques may be used to determine thermal uniformity assessments and/or confidence metrics associated with the temperature assessments. In some examples, a thermal uniformity assessment component 324 may determine the percentage of temperature readings in the bounding box for an individual, that are within a number or percentage of the maximum temperature reading. For instance, in FIG. 4A if the maximum in the first set of temperature readings 402 is 98.7° F., then the thermal uniformity assessment component 324 may determine a percentage (N %) of the first set of temperature readings 402 that fall within a predetermined range of the maximum reading (e.g., within 2.0° F., 2.5° F., 3.0° F., . . . ). The determined percentage (N %) that falls within range of the maximum temperature reading may be assigned by the thermal uniformity assessment component 324 as the thermal uniformity value.

As shown in FIGS. 4A-4C, the thermal uniformity values may be used to determine confidence levels associated with temperature assessments. In some case, a thermal uniformity value expressed as a percentage (e.g., a % of temperature readings within N degrees of the maximum reading) be used directly as the confidence value for the temperature assessment. In FIG. 4A, information box 404 indicates a confidence value of 64.7% in the temperature assessment, which may indicate that 64.7% of the set of temperature readings 402 are with a threshold range of the maximum reading of 98.7° F. Similarly, in FIG. 4B, information box 410 indicates a confidence value of 93.3%, which may indicate that 99.1% of the set of temperature readings 408 are with a threshold value of the maximum reading of 99.1° F., and in FIG. 4C, information box 416 indicates a confidence value of 85.7%, which may indicate that 85.7% of the set of temperature readings 414 are with a threshold value of the maximum reading of 103.4° F.

In other examples, the temperature assessment system 102 may use other techniques for determining thermal uniformity. In some cases, the thermal uniformity assessment component 324 may determine a temperature variation distribution based on the set of temperature readings for individual, and may calculate the thermal uniformity value based on the distribution. For instance, if a multi-modal distribution of temperature readings is detected, this distribution may indicate that first mode (or subset) of the temperature readings corresponds to the individual's skin, while other modes (or subsets) of the readings may correspond to the individual's hair, clothing, etc. In such instances, the temperature assessment component 322 and/or thermal uniformity assessment component 324 may analyze the characteristics of the multi-modal distribution (e.g., size of peaks, temperature ranges of peaks, corresponding locations and grouping within the visual image, etc.), and may select one or more of the modes within the multi-modal distribution to be used for performing the temperature assessment and/or thermal uniformity assessment.

As the above examples illustrate, thermal uniformity assessments (and/or confidence values) may be based on the distribution of the temperature readings associated with an individual. In some cases, narrow distributions may represent greater thermal uniformity and/or greater confidence in the temperature assessment for the individual, while wider distributions may represent less thermal uniformity and/or less confidence. Lower thermal uniformity values also may indicate that individual's skin is concealed to some degree by hair, clothing, masks, and/or other obstructions. For instance, the relatively low confidence value in FIG. 4A may be caused by the mask worn by the individual in the image 400, which may prevent some of the set of temperature readings 402 from being performed on the individual's skin. In contrast, FIG. 4B shows a relatively higher confidence value, which may be caused by the individual's lack of mask and/or other obstructions from hair, clothing, etc. In some cases, foreign objects that generate heat, such as wearable computing devices and hot beverages, also may cause lower thermal uniformity.

Objects worn on or around the individual, such as masks, face shields, clothing, hats, glasses, reflective jewelry, hair, electronic devices, and the like, may affect thermal uniformity assessments differently in different cases. When a temperature assessment system 102 performs a temperature assessment, any of these objects may be warmer than, cooler than, or the same as the individual's body temperature, and the temperatures of these objects may depend on various short-term factors or conditions. For instance, if the individual has recently entered the physical environment after being outdoors in warm weather, then the individual's hair and clothing may be warmer than his/her body temperature, whereas if the weather outside is cold then the then the individual's hair and clothing may be colder than his/her body temperature. Similarly, if the individual has put on a mask just before entering the physical environment, the mask may be near room temperature, but if the individual has been wearing the mask for a period of time then the mask may be warmer due to the individual's body heat and exhalations. In some examples, any or all of the components within the temperature assessment system 102 may be configured to detect optical patterns, thermal patterns, and/or combined optical-thermal patterns associated with various objects that may be worn by the individual. For instance, heuristics algorithms and/or machine-learning models may be generated and trained to detect that an individual is wearing a mask, face shield, hat, glasses, electronic earbuds, and/or various types of clothing or hairstyles that may affect the temperature readings within the individual's associated bounding box. When any of these objects or combination of objects is detected for an individual, the components of the temperature assessment system 102 may revise the assessment operations based on the detected objects. In some instances, the temperature assessment system 102 may mask and/or exclude the portions of the individual affected by any such detected objects when performing temperature assessments and/or thermal uniformity assessments.

As noted above, the temperature assessment system 102 may include an assessment evaluation component 328 to determine actions based on the results of individual assessments. For example, after determining a temperature assessment and an associated confidence value (e.g., based on a thermal uniformity assessment) for an individual, the assessment evaluation component 328 may compare the temperature assessment and/or confidence value to predetermined thresholds to determine whether or not to trigger a notification, whether or not to allow the individual to enter a restricted area, etc. As an example, if the assessed temperature of the individual is within an acceptable range (e.g., 97.0° F. to 100.0° F.) with a confidence above a threshold value, then the assessment evaluation component 328 may assign a benign status to the individual (e.g., “OK”) and may allow the individual to proceed without an alert, alarm, or secondary assessment. As another example, if the individual's assessed temperature is above the acceptable temperature range with a sufficient confidence level, then the assessment evaluation component 328 may assign the individual an unfavorable status (e.g., “Alert”) and may initiate one or more actions to trigger an alarm or notification, to prevent the individual from entering a restricted area, etc.

In some examples, based on the individual's assessed temperature and/or associated confidence level, the assessment evaluation component 328 may initiate a reassessment of the individual's temperature and/or thermal uniformity. For instance, if the confidence level (e.g., thermal uniformity) is below a threshold, and/or if the combination of the assessed temperature and confidence level do not fall with a combined threshold range, then the assessment evaluation component 328 may assign an unresolved status to the individual (e.g., “Pending”) and may perform one or more actions to initiate a reassessment of the individual. Such reassessments may be performed using some or all of the functionality described herein for components 314-326. In some cases, the assessment evaluation component 328 may instruct one or more of the components 314-326 to perform a reassessment of the individual, using the same techniques or different techniques. Even when reassessing an individual using the same assessment techniques and operations, the assessment results may be different based on the different position, different body posture and/or or angle of the individual with respect to the sensor systems 204. However, in some cases, the temperature assessment system 102 may perform multiple initial assessments, and/or assessments followed by reassessments, that use different temperature assessment and/or thermal uniformity assessment techniques. For instance, when performing multiple assessments/reassessments, the temperature assessment system 102 may determine different ROIs, different bounding boxes, and/or may use different combinations of sensor data for the different assessments. Additionally or alternatively, the temperature assessment system 102 may use different combinations algorithms or models when performing multiple assessments/reassessments. For instance, a first assessment of an individual may be performed using a first trained machine-learning model and a second assessment of the individual may be performed using a first trained machine-learning model and/or a heuristics-based algorithm.

In some implementations, the assessment evaluation component 328 may initiate reassessments of individuals in response to failing temperature and/or thermal uniformity assessments. For instance, in a scenario when the individual's clothing, hair, or an object worn by the individual affects the temperature readings within the bounding box of the individual, the scenario may fail a heuristics-based algorithm with a predetermined thresholds for temperature ranges and/or confidence values. In this scenario, the temperature assessment system 102 may initiate a pattern matching algorithm and/or trained machine learning model to detect one or more pieces of clothing, a hairstyle, and/or other object affecting the individual's temperature readings. When one or more such objects (e.g., hats, masks, glasses, electronic earbuds, etc.) can be detected using the trained model, the temperature assessment system 102 may be configured to determine the location of such objects, mask-out or exclude those portions of the individual's bounding box, and then initiate a reassessment of the individual with the modified bounding box. In some examples, a combination of optical and/or thermal sensor data may be used by machine learning models and/or heuristics-based pattern matching algorithms, to detect and exclude the patterns of such objects affecting the assessments of the individual.

Additionally or alternatively, when evaluating the results of temperature assessments and/or thermal uniformity assessments, the assessment evaluation component 328 may use historical assessment results retrieved from a log data store 218 or other storage. For instance, the temperature assessment system 102 may identify the particular individual being assessed, using various techniques such as facial recognition, biometrics, a badging system, etc. The identification of the individual may be performed as part of an assessment, or may be performed conditionally after an assessment based on the assessment results. After the individual has been identified, the assessment evaluation component 328 may retrieve and/or determine historical assessment records and/or patterns associated with the individual, and may use the historical records/patterns to evaluate the assessment results for the individual. For instance, if an individual has a previous pattern of elevated body temperature measurements, the assessment evaluation component 328 may increase an individual-specific temperature threshold for performing actions such as triggering alerts and notifications, denying access to restricted areas, etc. Temperature measurement patterns may apply to specific individuals and/or groups of individuals, and such patterns also may be associated with particular times, days, environmental conditions, etc. For instance, the assessment evaluation component 328 may determine that all individuals passing through the physical environment during certain times of the day have higher hair and clothing temperatures than during other times. The assessment evaluation component 328 may compensate accordingly to such patterns by adjusting the thresholds for assigning statuses, triggering notifications, restricting access, and performing other actions.

In addition to, or instead of, temperature assessments and thermal uniformity assessments, the temperature assessment system 102 also may perform mask assessments of individuals in the physical environment. During a mask assessment, the mask assessment component 326 may execute heuristic algorithms and/or machine learning models, using optical and/or thermal sensor data as input, to determine whether an individual detected in the environment is wearing a mask in compliance with a mask requirement. In some examples, the mask assessment component 326 may apply a machine-learning model configured to output a mask determination (e.g., mask or no mask) based on visual data, and then may verify the output of the visual machine learning model with a separate machine learning model or heuristics-based algorithm based on thermal data. A thermal machine learning model (or thermal heuristic algorithm) for detecting masks may be trained based on patterns of temperature readings on and around the mask area on the individual's face. When using thermal mask models/algorithms, the mask assessment component 326 may determine different temperature patterns for different types of masks and/or face shields. In examples that use one or more machine learning model(s) and/or algorithms based on both visual and thermal data, such examples may provide improved performance in distinguishing masks from objects such as beards and glasses. In contrast, algorithms and/or models based only on visual data may fail to distinguish masks from other such objects that partially obscure the individual's face. Additionally, models and/or algorithms based at least in part on thermal sensor data may identify masks and face shields which are transparent or have a face design, which might not be detected by algorithms or models based only on visual data.

As described above, the assessment evaluation component 328 may evaluate the results of temperature assessments and/or thermal uniformity assessments to determine statuses for the assessed individuals, and/or actions to perform via any of the various output devices or systems described herein. In various examples, any of the status determinations and/or output system controls determined by the assessment evaluation component 328 may be based on the results of mask assessments performed by the mask assessment component 326. For instance, information box 404 in FIG. 4A indicates that there is no alert and that the individual is permitted to access a restricted area, which may be based partially or entirely on the successful mask verification for the individual in image 400. In contrast, information box 410 in FIG. 4B indicates that the individual is not permitted to access a restricted area, which may be based partially or entirely on the failing mask verification for the individual in image 406. Finally, information box 416 in FIG. 4C indicates a successful mask verification for the individual in image 412, who is wearing a transparent face shield in compliance the mask requirement, but nonetheless indicates an alert and that the individual is not permitted to access a restricted area based on the individual's elevated body temperature.

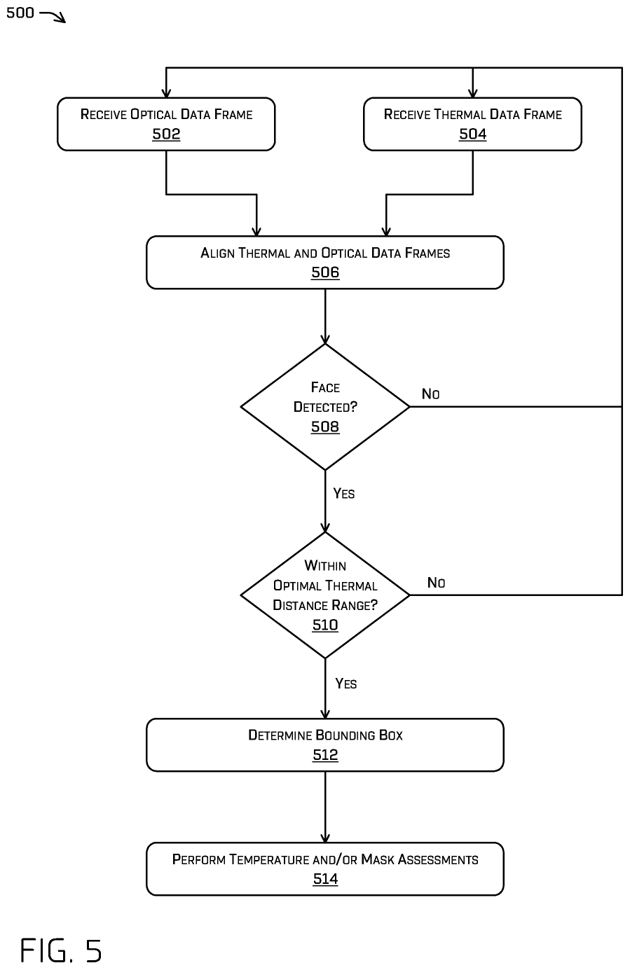

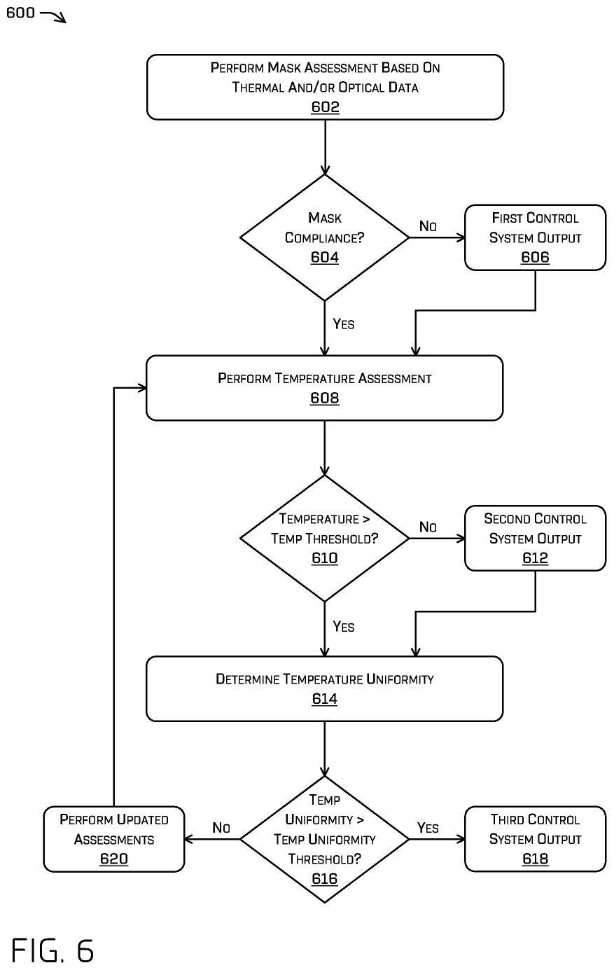

FIG. 5 is a flow diagram illustrating an example process 500 of performing assessments based on optical and/or thermal sensor data, and FIG. 6 is a flow diagram illustrating an example process 600 of controlling output systems based on the evaluations of temperature and/or mask assessments. As described below, the operations of process 500 and/or process 600 may be performed by one or more implementations of a temperature assessment system 102, alone and/or in conjunction with various related devices and/or systems described herein.

Processes 500 and 600 are each illustrated as a collection of blocks in a logical flow diagram, which represent a sequence of operations, some or all of which can be implemented in hardware, software, or a combination thereof. In the context of software, the blocks represent computer-executable instructions stored on one or more computer-readable media that, which when executed by one or more processors, perform the recited operations. Generally, computer-executable instructions include routines, programs, objects, components, encryption, deciphering, compressing, recording, data structures and the like that perform particular functions or implement particular abstract data types. The order in which the operations are described should not be construed as a limitation. Any number of the described blocks can be combined in any order and/or in parallel to implement the processes, or alternative processes, and not all of the blocks need be executed. For discussion purposes, the processes herein are described with reference to the frameworks, architectures and environments described in the examples herein, although the processes may be implemented in a wide variety of other frameworks, architectures or environments.

Referring now to FIG. 5, an example process 500 is shown for performing one or more assessments (e.g., temperature assessments, thermal uniformity assessments, and/or mask assessments) based on a combination of optical and/or thermal sensor data received from sensor systems 204 in a physical environment 202.

At operation 502, the temperature assessment system 102 receives optical data captured by one or more cameras or other optical sensors 206 operating in the physical environment 202. The temperature assessment system 102 may receive a single visual image frame representing a single point in time, or may receive a stream of image data from a video feed of the physical environment 202.

At operation 504, the temperature assessment system 102 receives associated thermal data from one or more thermal sensors 208 operating in the same physical environment 202. Similar to the optical data received in operation 502, the thermal data may include one or more data frames of representing a single point in time or a continuous data feed over a period of time.

At operation 506, the temperature assessment system 102 aligns the one or more optical and thermal data frames received in operations 502 and 504, to allow the combined optical/thermal data to be used in assessments. In some examples, the thermal/optical overlay component 320 of the temperature assessment system 102 may align the thermal and optical data by synchronizing the separate data frames based on timestamps, and overlaying the thermal/optical data frames based on angle and/or field of view. In some examples, the thermal/optical overlay component 320 may use an affine transformation to align the image frames.

At operation 508, the temperature assessment system 102 may analyze the aligned thermal/optical sensor data to determine if and when a human face is detected within the data. As described above, a facial detection component 316 may be configured to continually monitor one or more video feeds of the physical environment 202 to detect and/or recognize human faces within the environment. In various examples, operation 508 may be performed based only on the optical sensor data, only on the thermal data, or may use the combined thermal/optical data as well as data from other sensors in the sensor systems 304 (e.g., lidar/radar, infrared, microphones, biometrics, etc.) to detect or confirm the presence and location of the individual. In some instances, facial detection component 316 may use a first machine-learning model configured to detect faces within visual image data, and a second machine-learning model and/or heuristics-based algorithm configured to verify the facial detection based on thermal image patterns. When the facial detection component 316 does not detect a face based on the thermal/optical sensor data (508:No), process 500 may return to operations 502 and 504 to receive additional sensor data from optical and thermal image data feeds, respectively.

When an individual is detected by the facial detection component 316 based on the thermal/optical sensor data (508:Yes), then at operation 510 the temperature assessment system 102 may use a distance assessment component 318 to determine if the individual is within an optimal distance range for thermal scanning in the physical environment 202. In various examples, the distance assessment component 318 may use the image data and/or data from other sensors (e.g., lidar, radar, microphones, etc.), to determine the relative location of the individual detected in operation 508 with respect to the thermal sensors 208 in the environment. The optimal temperature scanning range for the thermal sensors 208 may be based on types and configurations of the sensors, as well as the conditions within the environment such as the ambient temperature, humidity, lighting, and wind. For example, when the distance assessment component 318 determines that the individual detected is either too close or too far from the thermal sensors 208 for accurate temperature measurements (510:No), process 500 may return to operations 502 and 504 to receive additional sensor data from optical and thermal image data feeds, respectively. However, when the distance assessment component 318 determines that the individual detected is within an acceptable (and/or optimal) range for performing accurate temperature measurements (510:Yes), process 500 may proceed to operations 512 and 514.

In some examples, the distance assessment component 318 also may determine orientation and/or direction of movement of the individual, and operation 510 may be based on these additional determinations. For example, if a detected individual is within an optimal range of the thermal sensors 208, but is moving across the field of view of the sensors and/or moving away from the restricted area, then the temperature assessment system 102 may determine that the individual should not to be assessed. However, if the individual is within the optimal thermal sensor range and is facing the thermal sensors 208 and/or moving toward the restricted area, then the temperature assessment system 102 may proceed to perform temperature and/or mask assessments of the individual.

At operation 512, the temperature assessment system 102 determines a bounding box associated with the individual. In various examples, the bounding box may be determined by the facial detection component 516, the distance assessment component 518, and/or assessment components 322-326. As described above, temperature assessment system 102 may determine the size and shape of the bounding box to include at least head and neck region of the detected individual. In some instances, the bounding box may be square or rectangular in shape, and in other instances a non-rectangular bounding boxes various sizes and shapes. In some cases, the temperature assessment system 102 may use various sensor data to determine a bounding box that corresponds to the outline of the detected individual. Additionally or alternatively, the temperature assessment system 102 may determine a bounding box that excludes certain shapes based on objects detected within the bounding box for which temperature measurements are not likely to be accurate measurements of the individual's body temperature. For instance, after determining a bounding box associated with the individual, the temperature assessment system 102 may use machine-learning models to detect certain regions and/or object types within the bounding box based on visual and/or thermal data (e.g., hair, hats, clothing, electronic devices, etc.), and may modify the bounding box to exclude the object shapes at the detected locations.

At operation 514, the temperature assessment system 102 performs one or more assessments of the individual based on the visual and/or temperature readings corresponding to the bounding box determined in operation 512. The assessments performed in operation 514 may include temperature assessments, thermal uniformity assessments, and/or mask assessments, and may involve any combination of the assessment techniques described above in connection with FIGS. 1-4C. The assessments performed in operation 514 may use a single heuristics-based algorithm, single machine-learning model, and/or a combination of multiple such algorithms and models. Additionally, the assessments performed in operation 514 may use as input data any combination of the optical sensor data, thermal sensor data, and/or other sensor data collected by the sensor systems 204 operating in the physical environment 202. As described above, one or more assessments also may be based on the results of previous assessments and/or historical data patterns relating to temperatures, mask compliance, and/or other assessment results patterns.

Referring now to FIG. 6, another example process 600 is shown for controlling one or more output systems based on the evaluations of temperature and/or mask assessments. As described below, the operations in process 600 may be performed by one or more components within a temperature assessment system 102, such as an assessment evaluation component 528 in conjunction with one or more output control systems. In some examples, the assessment evaluation component 528 may monitor and receive assessment results from assessment components 522-526, and may evaluate and determine individual statuses and/or actions to be initiated at output devices/systems based on the assessment results. Although process 600 illustrates an example set of ordered operations that a temperature assessment system 102 may perform to evaluate temperature and mask assessment results, it can be understood from the context of this disclosure that in other examples, the temperature assessment system 102 may perform other evaluations and/or in different orders to cause different actions to be actions to be performed at the output devices/systems.

At operation 602, the temperature assessment system 102 may performs a mask assessment based on the thermal and/or optical sensor data received from the sensor systems 204, for an individual detected within a physical environment 202. At operation 604, the temperature assessment system 102 evaluates the results of the mask assessment and determines whether the detected individual is in compliance with the mask requirement(s) of the physical environment and/or restricted area toward which the individual is moving. Operations 602 and 604 may be performed by the mask assessment component 326 and/or the assessment evaluation component 328, using any combination of the various mask assessment and/or mask verification techniques described herein.

If the detected individual is determined not to be wearing a mask and/or is not in compliance with the mask requirement(s) of the physical environment 202 (604:No), then at operation 606 the temperature assessment system 102 performs a first output system control. The output system controls described in this example may include control instructions transmitted from the temperature assessment system 102 via the output control system(s) 330, which may update a user interface, cause notifications to be transmitted to user devices, and/or may control various electronic device or systems within the physical environment 202. In some examples, operation 606 may include transmitting a control instruction to an output a visual or audio alert system in the physical environment 202 instructing the individual to wear a mask. In other examples, operation 606 may include initiating a facial recognition process or other biometric identification process to identify the individual, and/or controlling an electronic access system 220 to prevent the individual from accessing a restricted area.

In this example, if the detected individual is determined not to be in compliance with the mask requirement(s) (604:Yes), then at operation 608 the temperature assessment system 102 may perform a temperature measurement for the detected individual. Operation 608 may be performed by the temperature assessment component 322, using machine-learning models and/or heuristics-based algorithms, using any combination of the various temperature assessment techniques described herein.

At operation 610, the temperature assessment system 102 evaluates the results of the temperature assessment performed at operation 608 and determines whether the body temperature of the detected individual is exceeds a maximum temperature threshold. If the detected individual is assessed not to have a body temperature exceeding the maximum temperature threshold (610:No), then at operation 612 the temperature assessment system 102 performs a second output system control. The second output system control may include any combination of the various output instructions or controls or output devices/systems described herein, including initiating alerts, transmitting notifications, and/or controlling any of the output devices/systems described herein. In this example, based on the determination that the individual is in compliance with the mask requirement(s), and that the individual does not exceed the maximum temperature threshold, the second output system control(s) performed in operation 612 may include assigning the individual a benign status, updating a user interface to indicate no alert or alarm condition, and/or allowing the individual to access the restricted area.

In this example, if the detected individual is assessed to have a body temperature exceeding the maximum temperature threshold (610:Yes), then at operation 614 the temperature assessment system 102 determines a confidence value associated with the temperature assessment, by performing a thermal uniformity assessment. Operation 614 may be performed by the thermal uniformity assessment component 324, using machine-learning models and/or heuristics-based algorithms, using any combination of the various thermal uniformity assessment techniques described herein.

At operation 616, the temperature assessment system 102 evaluates the results of the thermal uniformity assessment performed at operation 614 and determines whether the thermal uniformity associated with the individual's temperature assessment exceeds a thermal uniformity threshold. If the thermal uniformity associated with the temperature assessment is greater than the thermal uniformity threshold (616:Yes), then at operation 618 the temperature assessment system 102 performs a third output system control. The third output system control may include any combination of the various output instructions or controls or output devices/systems described herein, including initiating alerts, transmitting notifications, and/or controlling any of the output devices/systems described herein. In this example, based on the determination that the individual's body temperature exceeds the maximum temperature threshold, and that the thermal uniformity assessment indicates a sufficiently high level of confidence in the temperature assessment, the third output system control(s) performed in operation 618 may include assigning the individual an unfavorable status, triggering one or more alarms or notification, and/or instructing an electronic access system 220 to deny the individual access to a restricted area.

In contrast, if the thermal uniformity associated with the temperature assessment does not exceed the thermal uniformity threshold (616:No), then at operation 620 the temperature assessment system 102 may initiate an updated temperature assessment and/or a thermal uniformity assessment for the individual. In this example, the evaluation of the assessments indicates a body temperature for the individual that exceeds the maximum temperature threshold, but a thermal uniformity value that does not indicate a sufficiently high level of confidence in the temperature assessment. As a result, the temperature assessment system 102 may initiate one or more additional assessments of the individual to determine an updated temperature measurement and/or an updated confidence in the temperature measurement. As described above, reassessments of an individual performed by the temperature assessment system 102 may use the same or different techniques for performing the temperature reassessment of the individual, using the same techniques (e.g., using updated sensor data) and/or different techniques (e.g., using different algorithms or models) from those used in the initial temperature assessments.

### CONCLUSION

While one or more examples of the techniques described herein have been described, various alterations, additions, permutations and equivalents thereof are included within the scope of the techniques described herein. As can be understood, the components discussed herein are described as divided for illustrative purposes. However, the operations performed by the various components can be combined or performed in any other component. It should also be understood, that components or steps discussed with respect to one example or implementation may be used in conjunction with components or steps of other examples.

In the description of examples, reference is made to the accompanying drawings that form a part hereof, which show by way of illustration specific examples of the claimed subject matter. It is to be understood that other examples can be used and that changes or alterations, such as structural changes, can be made. Such examples, changes or alterations are not necessarily departures from the scope with respect to the intended claimed subject matter. While the steps herein may be presented in a certain order, in some cases the ordering may be changed so that certain inputs are provided at different times or in a different order without changing the function of the systems and methods described. The disclosed procedures could also be executed in different orders. Additionally, various computations that are herein need not be performed in the order disclosed, and other examples using alternative orderings of the computations could be readily implemented. In addition to being reordered, the computations could also be decomposed into sub-computations with the same results.

Although the subject matter has been described in language specific to structural features and/or methodological acts, it is to be understood that the subject matter defined in the appended claims is not necessarily limited to the specific features or acts described. Rather, the specific features and acts are disclosed as example forms of implementing the claims.

The components described herein represent instructions that may be stored in any type of computer-readable medium and may be implemented in software and/or hardware. All of the methods and processes described above may be embodied in, and fully automated via, software code modules and/or computer-executable instructions executed by one or more computers or processors, hardware, or some combination thereof. Some or all of the methods may alternatively be embodied in specialized computer hardware.

Conditional language such as, among others, “may,” “could,” “may” or “might,” unless specifically stated otherwise, are understood within the context to present that certain examples include, while other examples do not include, certain features, elements and/or steps. Thus, such conditional language is not generally intended to imply that certain features, elements and/or steps are in any way required for one or more examples or that one or more examples necessarily include logic for deciding, with or without user input or prompting, whether certain features, elements and/or steps are included or are to be performed in any particular example.

Conjunctive language such as the phrase “at least one of X, Y or Z,” unless specifically stated otherwise, is to be understood to present that an item, term, etc. may be either X, Y, or Z, or any combination thereof, including multiples of each element. Unless explicitly described as singular, “a” means singular and plural.

Any routine descriptions, elements or blocks in the flow diagrams described herein and/or depicted in the attached figures should be understood as potentially representing modules, segments, or portions of code that include one or more computer-executable instructions for implementing specific logical functions or elements in the routine. Alternate implementations are included within the scope of the examples described herein in which elements or functions may be deleted, or executed out of order from that shown or discussed, including substantially synchronously, in reverse order, with additional operations, or omitting operations, depending on the functionality involved as would be understood by those skilled in the art.

Many variations and modifications may be made to the above-described examples, the elements of which are to be understood as being among other acceptable examples. All such modifications and variations are intended to be included herein within the scope of this disclosure and protected by the following claims.

What is claimed is:

1. A method comprising:
receiving, by a computing device, an optical image frame comprising a plurality of optical readings;
receiving, by the computing device, a thermal image frame comprising a plurality of thermal readings;
determining, by the computing device, a visual representation of a person at a first location within the optical image frame;
determining, by the computing device, a first region of the thermal image frame, based on the first location within the optical image frame;
determining, by the computing device, a temperature measurement for the person, based on one or more thermal readings within the first region of the thermal image frame;
comparing, by the computing device, the temperature measurement to a temperature threshold; and
controlling, by the computing device, an output system based at least in part on the comparison of the temperature measurement to the temperature threshold.

2. The method of claim 1, further comprising:
determining, by the computing device, a second visual representation of a second person at a second location within the optical image frame;
determining, by the computing device, a second region of the thermal image frame, based on the second location within the optical image frame; and
determining, by the computing device, a second temperature measurement for the second person, based on one or more thermal readings within the second region of the thermal image frame.

3. The method of claim 1, further comprising:
determining a thermal uniformity measurement associated with the temperature measurement for the person, based at least in part on the thermal readings within the first region of the thermal image frame; and
comparing the thermal uniformity measurement to a thermal uniformity threshold,
wherein controlling the output system is based at least in part on the comparison of the thermal uniformity measurement to the thermal uniformity threshold.

4. The method of claim 1, further comprising:
determining whether the person is in compliance with a mask requirement, based at least in part on the optical readings within the first location of the optical image frame,
wherein controlling the output system is based at least in part on the determination of whether the person is in compliance with the mask requirement.

5. The method of claim 1, wherein controlling the output system comprises at least one of:
performing a facial recognition operation based on the visual representation of the person;
transmitting a notification including the temperature measurement for the person, to a user device associated with the person; or
controlling an electronic access system to restrict access by the person to a physical location.

6. The method of claim 1, wherein determining the temperature measurement for the person comprises:
determining a first maximum temperature value based on the thermal readings within the first region of the thermal image frame.

7. The method of claim 6, wherein determining the temperature measurement for the person further comprises:
determining a first subset of the thermal readings within the first region of the thermal image frame that are within a predetermined temperature range of the first maximum temperature value;
determining a thermal uniformity measurement based at least in part on a number of thermal readings in the first subset;
comparing the thermal uniformity measurement to a thermal uniformity threshold; and
based at least in part on determining that the thermal uniformity measurement is not greater than the thermal uniformity threshold, determining a second maximum temperature value based on the thermal readings within the first region of the thermal image frame, excluding the first subset of the thermal readings.

8. The method of claim 1, further comprising:
determining a pixel size of the visual representation of the person within the optical image frame; and
determining a distance between the person and a thermal sensor, based at least in part on the pixel size of the visual representation of the person,
wherein determining the temperature measurement for the person is based at least in part on the distance between the person and the thermal sensor.

9. A system comprising:
one or more processors; and
one or more computer-readable media storing computer-executable instructions that, when executed, cause the one or more processors to perform operations comprising:
receiving an optical image frame comprising a plurality of optical readings;
receiving a thermal image frame comprising a plurality of thermal readings;
determining a visual representation of a person at a first location within the optical image frame;
determining a first region of the thermal image frame, based on the first location within the optical image frame;
determining a temperature measurement for the person, based on one or more thermal readings within the first region of the thermal image frame;
comparing the temperature measurement to a temperature threshold; and
controlling an output system based at least in part on the comparison of the temperature measurement to the temperature threshold.

10. The system of claim 9, the operations further comprising:
determining a second visual representation of a second person at a second location within the optical image frame;
determining a second region of the thermal image frame, based on the second location within the optical image frame; and
determining a second temperature measurement for the second person, based on one or more thermal readings within the second region of the thermal image frame.

11. The system of claim 9, the operations further comprising:
determining a thermal uniformity measurement associated with the temperature measurement for the person, based at least in part on the thermal readings within the first region of the thermal image frame; and
comparing the thermal uniformity measurement to a thermal uniformity threshold,
wherein controlling the output system is based at least in part on the comparison of the thermal uniformity measurement to the thermal uniformity threshold.

12. The system of claim 9, the operations further comprising:
determining whether the person is in compliance with a mask requirement, based at least in part on the optical readings within the first location of the optical image frame,
wherein controlling the output system is based at least in part on the determination of whether the person is in compliance with the mask requirement.

13. The system of claim 9, wherein controlling the output system comprises at least one of:
performing a facial recognition operation based on the visual representation of the person;
transmitting a notification including the temperature measurement for the person, to a user device associated with the person; or
controlling an electronic access system to restrict access by the person to a physical location.

14. The system of claim 9, wherein determining the temperature measurement for the person comprises:
determining a first maximum temperature value based on the thermal readings within the first region of the thermal image frame.

15. The system of claim 14, wherein determining the temperature measurement for the person further comprises:
determining a first subset of the thermal readings within the first region of the thermal image frame that are within a predetermined temperature range of the first maximum temperature value;
determining a thermal uniformity measurement based at least in part on a number of thermal readings in the first subset;
comparing the thermal uniformity measurement to a thermal uniformity threshold; and
based at least in part on determining that the thermal uniformity measurement is not greater than the thermal uniformity threshold, determining a second maximum temperature value based on the thermal readings within the first region of the thermal image frame, excluding the first subset of the thermal readings.

16. The system of claim 9, the operations further comprising:
determining a pixel size of the visual representation of the person within the optical image frame; and
determining a distance between the person and a thermal sensor, based at least in part on the pixel size of the visual representation of the person,
wherein determining the temperature measurement for the person is based at least in part on the distance between the person and the thermal sensor.

17. A thermal and optical data analysis system comprising:
a processing unit comprising one or more processors; and
memory storing a plurality of computer-executable components that, when executed, cause the one or more processors to perform computer-executable operations, the components comprising:
a thermal data component configured to receive a stream of thermal data from a thermal sensor;
an optical data component configured to receive a stream of optical data from an optical sensor;
a facial detection component configured to detect a visual representation of a person within the stream of optical data;
a distance assessment component configured to determine a distance between the person and the thermal sensor;
a temperature assessment component configured to determine a temperature measurement associated with the person, based on the thermal data;
a thermal uniformity assessment component configured to determine a thermal uniformity measurement associated with the temperature measurement;
a mask assessment component configured to determine a mask status associated with the person; and
an output system control component configured to control an output system based at least in part on the temperature measurement associated with the person, the thermal uniformity measurement associated with the temperature measurement, and the mask status associated with the person.

18. The thermal and optical data analysis system of claim 17, wherein the output system comprises an electronic access system controlling access to a physical location.

19. The thermal and optical data analysis system of claim 17, wherein the output system comprises a monitoring system configured to output a video feed based on the thermal data and the optical data.

20. The thermal and optical data analysis system of claim 17, wherein the output system comprises a notification system configured to transmit a notification including the temperature measurement associated with the person and the mask status associated with the person.
